(19) United States
(12) Patent Application Publication
(10) Pub. No.: US 2007/0287632 A1
(43) Pub. Date: Dec. 13, 2007

(54) Sulphur-Containing Oils For Controlling Plant Pathogens And Stimulating Nutrient Uptake

(76) Inventor: Anton Pohoreski, Boroko (PG)

Correspondence Address: COOLEY GODWARD KRONISH LLP;ATTN: Patent Group, Suite 1100, 777 - 6th Street, NW, WASHINGTON, DC, 20001, US

(21) Appl. No.: 10/594,510

(22) PCT Filed: Mar. 31, 2005
(86) PCT No.: PCT/IB05/00850

§ 371 Date: Sep. 28, 2006

Related U.S. Application Data
(60) Provisional application No. 60/557,676, filed on Mar. 31, 2004.

(51) Int. Cl. A01N 59/02; A01N 31/00; A01P 3/00; C07C 309/62

(52) U.S. Cl. 504/189; 424/776; 504/349; 514/706; 514/708; 554/85; 554/98

(57) ABSTRACT

The present invention relates to sulphur-containing oils, their preparation, and their use in controlling/killing plant pathogens and stimulating a plant's uptake of essential elements from the soil and to act as a soil pH adjuster.

### FIELD OF THE INVENTION

The present invention relates to various sulphur-containing oils, their preparation and their use to kill and/or control most types of plant pathogens such as fungi, mildews, molds, mosses and rusts in and on plants, in soil or water and on artificial surfaces. The described sulphur-containing oils may also be used to stimulate a plant's uptake of essential elements from the soil and to act as a soil pH adjuster.

### BACKGROUND OF THE INVENTION

Agrochemical compounds currently used to control plant pathogens tend to exhibit varying degrees of toxicity toward humans, the environment, the host plants and the soil. Given however that the agricultural industry experiences significant annual losses in crop yields, plant damage and premature plant death due to soil and plant fungi, mildews, molds, mosses and rusts, there is a constant effort to discover safer, more selective, more potent and more environmentally friendly products.

It has long been known that elemental sulphur possesses excellent fungicidal properties but that it also has some major drawbacks that prevents its large-scale commercial use in the field. For one thing, sulphur is generally phytotoxic to most plants. In addition, sulphur's weather sensitivity (i.e., it's susceptibility to conditions of heat and humidity), may exacerbate its phytotoxic properties. A further disadvantage of using sulphur is that it cannot be applied during certain stages of plant growth, such as flowering, because it typically destroys the flower and/or resulting fruit. For example, applications of elemental sulphur during the flowering stage of hops (Humulus spp.) causes bloom burn, leading to reduced yields.

Because of these disadvantages associated with the use of elemental sulphur as well as the problems inherent in the use of other plant pathogen control agents, various alternative agents and methods have been employed in an attempt to control pathogens such as fingi, which exist on plants, in the soil and in sources of water. For example, essential oils and plant oils have been used as control agents with limited success. A major drawback to the use of oils, however, is their tendency to plug plant stomata; resulting in stunted growth or death, typically within 48 hours after application.

The problems encountered with the individual use of elemental sulphur and essential oils have been addressed by the present invention which provides an effective method of producing plant pathogen control agents from the combination of sulphur and various oils.

### SUMMARY OF THE INVENTION

The present invention relates to a sulphur-containing oil that combines the anti-pathogenic activity of elemental sulphur with the wettability properties of any of several oils.

An aspect of the invention is a process of preparing a water-soluble sulphur-containing oil, comprising the steps of combining an oil containing at least one sulphur-reactive site selected from the group consisting of an alkene bond, an alkyne bond and a hydroxy group with a sulphur-donating agent under conditions sufficient to cause at least about 30% of the sulphur-reactive sites in the oil to react with the sulphur-donating agent; allowing the resulting sulphur-containing oil to stand for a time sufficient to complete reaction with the sulphur-donating agent; and neutralizing the sulphur-containing oil with a base.

In a preferred embodiment, the sulphur-donating agent is selected from the group consisting of sulphuric acid, sulphur trioxide and sodium bisulfite.

In a more preferred embodiment, the sulphur-donating agent is sulphuric acid.

In a more preferred embodiment, the concentration of the sulphuric acid is at least about 2 N.

In a preferred embodiment, the sulphur-containing oil is selected from the group consisting of a sulphated oil, a sulphonated oil and mixtures thereof.

In a more preferred embodiment, the sulphur-containing oil is a sulphated oil.

In a preferred embodiment, the base is selected from the group consisting of sodium hydroxide, potassium hydroxide, sodium carbonate, potassium carbonate, calcium hydroxide, diethanolamine and ammonium hydroxide.

In a more preferred embodiment, the base is sodium hydroxide.

In a more preferred embodiment, the concentration of the sodium hydroxide is at least about 2 N.

In a preferred embodiment, the oil is selected from the group consisting from canola, coconut, palm, cottonseed, palm kernel, olive, flax, castor, soybean, sunflower, corn, grape seed and peanut.

In a more preferred embodiment, the oil is canola oil.

In a preferred embodiment, the oil and the sulphur-donating agent are combined under cooling, wherein the cooling maintains the reaction temperature below about 40° C.

In a preferred embodiment, the neutralization of the sulphur-containing oil with the base occurs under cooling, wherein the cooling maintains the reaction temperature below about 40° C.

Another aspect of the invention is a fungicidal, water-soluble sulphur-containing oil prepared by combining an oil containing at least one sulphur-reactive site selected from the group consisting of an alkene bond, an alkyne bond and a hydroxy group with a sulphur-donating agent under conditions sufficient to cause at least about 30% of the sulphur-reactive sites in the oil to react with the sulphur-donating agent; allowing the resulting sulphur-containing oil to stand for a time sufficient to complete reaction with the sulphur-donating agent; and neutralizing the sulphur-containing oil with a base.

Another aspect of the invention is a method of controlling or killing a plant pathogen present in a plant, in soil, in water or on an artificial surface comprising contacting the pathogen with an effective amount of a sulphur-containing oil of the invention.

In a preferred embodiment, the pathogen is selected from the group consisting of molds, mildews, fungi, mosses and rusts.

In a preferred embodiment, the artificial surface is a roof.

Another aspect of the invention is a method of stimulating a plant's uptake of nutrients from the soil, comprising treating a plant in need thereof with an effective amount of a sulphur-containing oil of the invention.

Another aspect of the invention is a method of adjusting soil pH comprising treating the soil with an amount of the sulphur-containing oil of the invention effective to obtain a contemplated pH. For example, application of an acidic formulation of a sulphur-containing oil of the present invention will decrease soil pH. Conversely, application of a basic formulation of a sulphur-containing oil of the invention will increase the soil pH. Because different plants have different preferences for the pH of the soils in which they exist, the formulation of an applied sulphur-containing oil can be customized to the particular plant species being treated.

### BRIEF DESCRIPTION OF THE DRAWINGS

FIG. 1 depicts various methods of sulfatation and sulfonation of generically structured oils.

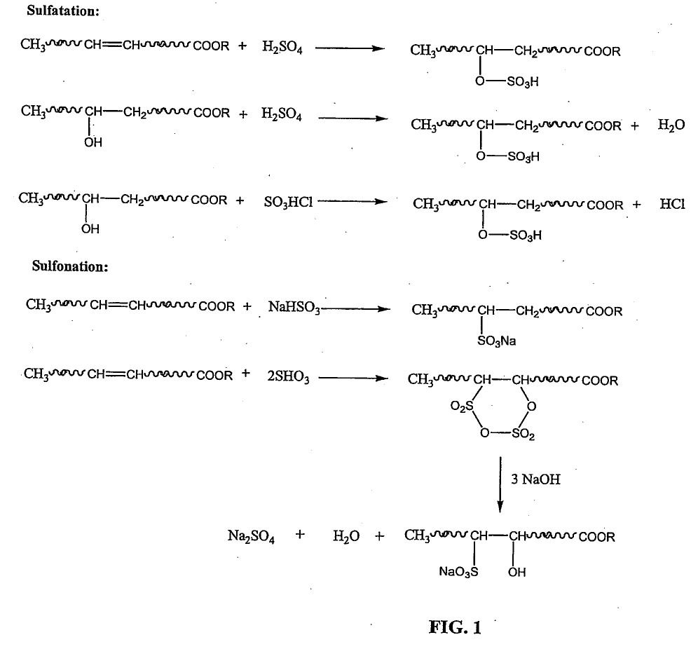

FIG. 2 depicts the analysis of selected elements in plant tissue from a plant not treated with the sulphur-containing oils of the invention.

FIG. 3 depicts the analysis of selected elements in plant tissue from a plant that has been treated with a sulphur-containing oil of the invention.

### DETAILED DESCRIPTION OF THE INVENTION

The reaction of sulphur or a sulphur-donating agent with any of a number of suitable oils results in a sulphur-containing oil of the present invention wherein the phytotoxicity of the sulphur is attenuated compared to its uncombined state, and the oil, which was previously water-insoluble, becomes substantially water-soluble, thus eliminating the risk of injury to the plant by the oil's plugging of the plant's stomata.

In one embodiment of the invention, a predetermined amount of a suitable oil is added to a reactor equipped both with a means for cooling the reactor and a means for agitating the contents of the reactor. While the oil is being agitated, a predetermined amount of a sulphur-donating agent, preferably sulphuric acid, more preferably at least about 3 N sulphuric acid, and even more preferably about 6 N concentrated sulphuric acid, is slowly added to the oil. Because the reaction of the oil with the sulphur-donating agent is exothermic, the employment of a means for cooling the reactor is typically required. In an embodiment of the invention, the oil to be treated with the sulphur-donating agent is dissolved in a solvent. In a preferred embodiment, the oil is neat, i.e., without solvent present.

The sulphur-containing agent is added at such a rate that the reaction temperature does not exceed the temperature beyond which the oil and the sulphur-containing agent begin to decompose. In one embodiment of the invention, the reaction temperature does not exceed about 40° C. In a more preferred embodiment, the reaction temperature does not exceed about 35° C. If the temperature is too high, the oil may become carbonized, and where sulphuric acid is used as the sulphur-containing agent, undesired reduction to sulphur anhydride may occur. The rate of addition of the sulphur-containing agent to the oil is important, as addition that is either too slow or too rapid may result in undesired fluctuations in the reaction temperature that leads to a product with reduced anti-pathogenic efficacy. The minimum effective temperature at which the sulphur-containing agent is added to the oil is about 10° C. In a more preferred embodiment, the minimum temperature at which the sulphur-containing agent is added to the oil is about 20° C. In even more preferred embodiments, the minimum temperature at which the sulphur-containing agent is added to the oil is about 25° C. or about 30° C.

After all of the sulphur-containing acid has been added to the oil, the reaction mixture is allowed to stand for a predetermined period of time, preferably anywhere from about 1 to about 48 hours, to allow completion of the sulfating process (i.e. incorporation of sulphur into the oil). It was further discovered that the sulphated oil may be allowed to stand much longer than 48 hours without significant decomposition. In a preferred embodiment, at least about 30% of the sulphur-active sites present in the oil have reacted with the sulphur-donating agent. In a more preferred embodiment, at least about 50% of the sulphur-active sites present in the oil have reacted with the sulphur-donating agent. In an even more preferred embodiment, at least about 70% of the sulphur-active sites present in the oil have reacted with the sulphur-donating agent. Thus, the present invention contemplates the percentage of sulphur-active sites present in the oil that have reacted with the sulphur-donating agent as being at least about 30% or at least about 40%, or at least about 45%, or at least about 50%, or at least about 55%, or at least about 60%, or at least about 65%, or at least about 70%. Percentages about 70% may also prove to be beneficial to fungicidal activity and are contemplated by this disclosure of the present invention. If less than about 30% of the sulphur-active sites present in the oil have reacted with the sulphur-donating agent to incorporate sulphur into the oil, the resulting oil is not very water soluble and tends to plug a plant's stomata, resulting in the death of the plant.

To compensate for this general lack of water solubility, various surfactants may be used in combination with the oil. Suitable surfactants include, but are not limited to, polyethoxylated alcohols (e.g. Igepal CO-630), polyethoxylated alkylphenols, polyethoxylated sorbitan fatty acid esters (e.g., Tween 80), dialkyl sulfosuccinates, alkyl sulfates, alkylbenzene sulfonates, organosilicones, N,N-dialkyltaurates, lignin sulfonates, naphthalene sulfonate formaldehyde condensates, polycarboxylates and polyoxyethylene/polyoxypropylene block copolymers.

Solid diluents may also be included in a formulation of the sulphur-containing oils of the invention. Suitable solid diluents include, for example, ground corn cobs, clays such as bentonite, montmorillonite, attapulgite and kaolin, starch, sugar, silica, talc, diatomaceous earth, urea, calcium carbonate, sodium carbonate and bicarbonate and sodium sulfate.

Liquid diluents other than water may be included in a formulation of the sulphur-containing oils. Suitable liquid diluents include, for example, alcohols and glycols.

The sulphur-containing oils may also be mixed with one or more other insecticides, fungicides, nematocides, bactericides, acaricides, growth regulators, chemosterilants, semiochemicals, repellents, attractants, pheromones, feeding stimulants or other biologically active compounds to form a multi-component pesticide that provides an even broader spectrum of agricultural protection.

As the reaction mixture is being agitated, a predetermined amount of a base, such as, for example, sodium hydroxide, is slowly added. Because this neutralization process is also exothermic, a means for cooling the reactor may be required. In one embodiment of the invention, the base is added at such a rate that the reaction temperature does not exceed about 40° C. In a more preferred embodiment of the invention, the reaction temperature does not exceed about 35° C. In an embodiment of the invention, the concentration of the base can range from about 1 N to about 6 N, with a preferred range of about 2 N to about 4 N, and with a more preferred concentration of about 3 N.

After the base has been added in its entirety to the reaction mixture, the resulting neutralized mixture is allowed to stand and to separate into at least two layers. The sulphur-containing oil layer typically forms the top layer and the aqueous layer containing the neutralized acid salts forms the bottom layer. For example, where sulphuric acid is the sulphur-containing acid used in the reaction and sodium hydroxide is the neutralizing agent, the neutralized acid salts in the bottom layer would largely consist of a supersaturated solution of sodium sulphate. This bottom aqueous layer is removed from the sulphur-containing oil layer. The pH of the separated sulphur-containing oil layer is then adjusted with a base, such as, for example, sodium hydroxide, to about 2 to about 8, more preferably from about 3 to about 6, and even more preferably from about 4 to about 5, depending on the type of application for which the sulphur-containing oil is to be used.

Prior to the neutralization step, the reaction mixture is optionally washed with an aqueous salt solution such as, for example, a sodium chloride solution, as an additional purification step. Similarly, after the neutralization step, the separated sulphur-containing oil layer is optionally washed with an aqueous salt solution.

Suitable oils that may be used in the invention include naturally occurring oils, fats or base stock synthesized from petroleum hydrocarbons. Examples of naturally occurring oils include, but are not limited to, canola oil, coconut oil, palm oil, cottonseed oil, palm kernel oil, olive oil, flax oil, castor oil, soybean oil, sunflower oil, corn oil, grape seed oil, peanut oil and mixtures thereof. Examples of fats include, but are not limited to, beef, sheep, bird, neats foot, herring, cod liver, seal and mixtures thereof. Preferred oils of the invention include canola oil, flax oil and soybean oil. A more preferred oil is canola oil. Variations in the purity of the starting oils may require minor modification of the procedures described herein to produce the sulphur-containing oils of the present invention. For example, starting oils that have been pretreated with acids other than, for example, sulphuric acid (e.g., phosphoric acid) may result in a less efficacious product.

Other classes of compounds may also be used to form the sulphur-containing oils of the invention such as, for example, oleic acid derivatives, such as, for example, methyl oleate, oleyl oleate, glycerol trioleate and propylene glycol dioleate; ricinoleate esters; and partial glycerols. In general, any product that contains unsaturated fat chains (i.e., alkene (carbon-carbon double bonds) and or alkyne (carbon-carbon triple bonds)) or at least one hydroxyl group on a fat chain and that can be made to incorporate sulphur may be used in the invention.

As used herein, a sulphur-donating agent is any compound that provides a sulphur atom, in any of its unoxidized or oxidized forms, for incorporation into another compound. Sulphur-donating agents include, for example, sulfating agents and sulphonating agents. In a preferred embodiment, sulphuric acid is the sulphur-donating agent.

As used herein, bases include, for example, an alkali or alkaline earth metal hydroxide, an alkali or alkaline earth metal carbonate or alkyl amines. Preferred embodiments include sodium hydroxide, potassium hydroxide, sodium carbonate, potassium carbonate, calcium hydroxide, diethanolamine and ammonium hydroxide. A more preferred base is sodium hydroxide.

The sulphur-containing oils of the invention may be produced in a continuous process or in batch, depending on the end product desired. In a continuous process, the sulphur-donating agent is sprayed into an atomization of the oil in a tower. In a batch process, the sulphur-donating agent is added to the oil in a mixing tank.

Sulphated oils and sulphonated oils represent preferred embodiments of the sulphur-containing oils of the present invention. Sulphated oils may be formed by treating a suitable oil with a sulfating agent, such as sulphuric acid or sulphur trioxide, while sulfonated oils may be formed by treating a suitable oil with a sulphonating agent, such as sodium bisulfite.

The compositions and methods of the present invention can be used to treat any applicable plant pathogen on any plant species in need of such treatment, thereby reducing the growth, numbers and/or detrimental effects of the pathogen on the plant. Plants suitable for treatment include, for example, any plant grown for any commercial purpose, including, but not limited to the following purposes: seed production, hay production, ornamental use, fruit production, berry production, vegetable production, oil production, protein production, forage production, animal grazing, golf courses, lawns, flower production, landscaping, erosion control, green manure, improving soil tilth/health, producing pharmaceutical products/drugs, producing food or food additives, smoking products, pulp production and wood production. Plants which are of interest for treatment are those which are colonized by pathogenic organisms and include flowering plants, grasses, including bent grass, vegetables, cereals and fruits including tomato, potato, artichoke, strawberries, corn, cereal grains, onion, cucumber, lettuce, tobacco, and citrus such as orange, lemons, limes and grapefruit, as well as bell peers and grapes, and fruit trees such as peach, apple and cherry, ornamentals such as roses and trees, particularly conifers. Also included are crops intended for consumption by fish, fowl and animals, including humans, directly or indirectly.

Examples of plants to treat using the compositions and methods of the present invention include, but are not limited to barley (Hordeum spp.), alfalfa (Medicago spp.), clover (Trifolium spp.), amaranth (Amaranthus spp.), bluegrass (Poa spp.), bean (Phaseolus spp.), bermudagrass (Cynodon spp.), cotton (Gossypium spp.), ryegrass (Lolium spp.), fescue (Festuca spp.), hop (Humulus spp.), lentil (Lens spp.), lupine (Lupinis spp.), marigold (Tagetes spp.), mint (Mentha spp.), oat (Avena spp.), peanut (Arachis spp.), potato (Solanum spp.), sugarcane (Saccharum spp.), tomato (Lycopersicon spp.), vetch (Vicia spp.), winged-bean (Psophocarpus spp.), zoysiagrass (Zoysia spp.), apple (Malus spp.), pear (Pyrus spp.), grape (Vitis spp.), strawberry (Fragaria spp.), hemlock (Tsuga spp.), poplar (Populus spp.), pine (Pinus spp.), zinnia (Zinnia spp.) and blackberry (Rubus spp.).

Examples of fungi to control or eliminate using the compositions and methods of the present invention include, but are not limited to the following Alternaria (Alternaria brassicola; Alternaria solani), Ascochyta (Ascochyta pisi); Botrytis (Botrytis cinerea); Cercospora (Cercospora kikuchii; Cercospora zeae-maydis); Colletotrichum (Colletotrichum lindemuthianum); Diplodia (Diplodia maydis); Erysiphe (Erysiphe graminis f. sp. graminis; Erysiphe graminis f. sp. hordei); Fusarium (Fusarium nivale; Fusarium oxysporum; Fusarium graminearum; Fusarium culmorum; Fusarium solani; Fusarium moniliforme; Fusarium roseum); Gaeumanomyces (Gaeumanomyces graminis f, sp. tritici); Helminthosporium (Helminthosporium turcicum; Helminthosporium carbonum; Helminthosporium maydis); Macrophomina (Macrophomina phaseolina); Magnaporthe (Magnaporthe grisea); Nectria (Nectria haematococca); Peronospora (Peronospora manshurica; Peronospora tabacina); Phoma (Phoma betae); Phymatotrichum (Phymatotrichum omnivorum); Phytophthora (Phytophthora cinnamomi; Phytophthora cactorum; Phytophthora phaseoli; Phytophthora parasitica; Phytophthora citrophthora; Phytophthora megasperma f. sp. sojae; Phytophthora infestans); Plasmopara (Plasmopara viticola); Podosphaera (Podosphaera leucotricha); Puccinia (Puccinia sorghi; Puccinia striiformis; Puccinia graminis f. sp. tritici; Puccinia asparagi; Puccinia recondita; Puccinia arachidis); Pyrenophora (Pyrenophora tritici-repentis); Pyricularia (Pyricularia oryzae); Pythium (Pythium aphanidermatum; Pythium ultimum); Rhizoctonia (Rhizoctonia solani; Rhizoctonia cerealis); Sclerotium (Sclerotium rolfsii); Sclerotinia (Sclerotinia sclerotiorum); Septoria (Septoria lycopersici; Septoria glycines; Septoria nodorum; septoria tritici); Thielaviopsis (Thielaviopsis basicola); Uncinula (Uncinula necator); Venturia (Venturia inaequalis); and Verticillium (Verticillium dahliae; Verticillium albo-atrum).

Of particular interest is treatment of plants affected by powdery mildew which is caused by target organs which are species of fungi of the family Erysiphaceae. Generally the genera are distinguished from each other by the number (one as opposed to several) of asci per cleistotheciun and by the morphology of hypal appendages growing out of the walls of the creistothecium. As an example the following genera cause powdery mildew in the indicated plants: Erysiphe cichoracearum, begonia, chrysanthemum, cosmos, cucurbits, dahlia, flax, lettuce and zinnia; E. graminis, with cereals and grasses; E. polgoni, beans, soybeans, clovers, and other legumes, beets, cabbage and other crucifers, cucumber and cantaloupe, delphinium and hydrangea; Microsphaera alni, blueberry, catalpa, elm, lilac, oak, rhododendron, and sweet pea; Phyllactinia spp. catalpa, elm, maple and oak; Podosphaera leucotricha, apple, pear and quince; P. oxyacanthae, apricot, cherry, peach and plum; Spaelrotheca macularis, strawberries; S. mors-uvae, gooseberry and currant; S. pannosa, peach and rose; and Uncinula necator, grape, horse chestnut and linden. See, for example, U.S. Pat. No. 6,251,951.

Examples of rusts to control or eliminate using the compositions and methods of the present invention include, but are not limited to plants affected by rust caused by Basidiomycetes of the order Uredinales; Puccinia (P. graminis, P. stiiformis, P. recondita, P. hordei, P. coronata, P. sorghi., P. polysora, P. purpurea, P. sacchari P. kuehnii, P. stakmanii, P. asparagi, P. chrysanthemi, P. malvacearum, and P. antirrhini); Gymnosporangium (G. juniperi-virginianae, G. globosum); Hemileia (H. vastatrix) Phragmidium; Uromyces (U. caryophyllinus); Cronartium (C. ribicola, C. quercuum f. sp. fusiforme, C. quercuum f. sp. virginianae, C. comptoniae, C. comandrae, C. strobilinum); Melampsora (M. lini); Coleosporium (C. asterinum); Gymnoconia; Phakopsora (P. pahyrhizi) and Tranzschelia.

Treatments may be carried out daily, weekly or monthly. Any treatment schedule may be followed as long as optimal inhibition of fungal growth fungal disease is obtained. In the methods and products of the present invention, the step of contacting the sulphur-containing oils or their compositions may be carried out by any of the techniques known in the art. A preferred contacting method useful in the methods and products of the present invention is spraying.

In an embodiment of the invention, the sulphur-containing oils are diluted with water before being applied to the plants, soil, water or artificial surfaces to be treated. In a preferred embodiment, approximately 2 quarts of the sulphur-containing oils mixed with approximately 100 gallons of water is adequate for effectively treating about 1 acre of cropland. In another preferred aspect, the sulphur-containing oil is diluted approximately 1 part oil to approximately 25 parts water and then sprayed onto lawn grass or onto trees.

In another embodiment of the invention, the sulphur-containing oils are diluted with water and then sprayed onto artificial surfaces, such as roofs, for the control of, for example, mosses and molds.

In another embodiment of the invention, the sulphur-containing oils are diluted approximately 1 part oil to approximately 25 parts water and then contacted with soil such that the aqueous solution of the sulphur-containing oil is allowed to percolate through the soil, contacting roots, soil particles, etc.

In another embodiment of the invention, the sulphur-containing oils are used to treat aqueous-based plant pathogens. In a preferred aspect, the sulphur-containing oil is lightly sprayed onto the surface of a dry foodstuff that is to be fed to aquatic organisms such as aquatic mammals, fish and shrimp. The treated foodstuff is then fed to the organisms.

The amounts of the sulphur-containing oils necessary for effective treatment will, of course, vary depending on the type and severity of the problem.

The process described for preparing the sulphur-containing oils of the present invention appears to be important for the oil's fungicidal properties. As an example, a commercial sulphated canola oil (L. V. Loomis) was obtained, diluted with water, adjusted to a pH of 7 and sprayed onto hops infected with powdery mildew. No inhibition of the mildew was observed. Adjustment of the pH of the oil to 4 did not improve the oil's fungicidal properties.

### EXAMPLES

### Example 1

### General Preparation of a Sulphated Canola Oil

Raw canola oil (3 kg) was added to a double-walled vessel with cooling water capacity. Concentrated sulphuric acid (477 mL) was slowly added to the oil with stirring and cooling of the vessel via circulation of cool water. The sulphuric acid was added at such a rate that the temperature of the reaction mixture remained between about 30 to about 35° C. The addition was typically complete after about 2 hours. The reaction mixture was then allowed to stand for about 18 hours for the sulphation to proceed. Approximately 3 N sodium hydroxide (3,880 mL) was slowly added to the oil/acid reaction mixture with stirring and cooling of the vessel via circulation of cool water. The sodium hydroxide was added at such a rate that the temperature of the reaction mixture remained between about 30 to about 40° C. The addition was typically complete after about 2 hours. The neutralized reaction mixture was then allowed to sit for about 24 hours to allow the sulphated oil phase to rise to the top of the vessel and for the super saturated sodium sulphate water phase to settle to the bottom of the vessel. The lower water phase was discarded. The pH of the sulphated canola oil phase was less than about 1. The sulphated oil was diluted with R.O. (reverse osmosis) water at a ratio of 1 part oil to 4 parts water. At the same time, the pH of the resulting solution was adjusted to a pH of about 4 to about 5 with approximately 3 N sodium hydroxide. This particular formulation, i.e., a 20% solution of the sulphated canola oil in water at a pH of about 4 to about 5 was referred to during experimental field trials as “Sulplex.”

### Example 2

### Comparison of Raw Canola Oil with Sulphated Canola Oil

A comparison between raw (i.e., unsulphated) canola oil and sulphated canola oil as shown in Table 1 indicates that the C16:1, C18:2 and C18:3 double bond sites are positions of sulphur attachment. For example, the C18:2 double bond makes up 18.57% of the raw canola oil but only 1.44% of the sulphated canola oil—i.e., most of the C18:2 double bond has been sulfated. Similarly, the C18:3 double bond makes up 8.18% of the raw canola oil but only 0.17% of the sulphated canola oil. In measuring sulphur uptake, it was observed that raw canola oil contained only about 22.8 ppm of sulphur, whereas after sulphation, the canola oil contained about 2,340 ppm of sulphur.

TABLE 1
Comparison of Raw Canola Oil vs. Sulphated Canola Oil (“Sulplex”)

| Analysis | Raw | Sulphated |
|---|---|---|
| Sulphur | 22.8 ppm | 2,340.0 ppm |
| Peroxide Value | 2.32 meq/kg | 0.06 meq/kg |
| Free Fatty Acids | 0.7% | 45.9% |
| Fatty Acid Composition | | |
| C-14 | 0.06% | 0.13% |
| C-16 | 4.22 | 9.99 |
| C-16:1 | 0.27 | 0.25 |
| C-18 | 2.09 | 5.09 |
| C-18:1 | 62.97 | 65.29 |
| C-18:2 | 18.57 | 1.44 |
| C-18:3 | 8.18 | 0.17 |
| C-20 | 0.70 | 1.83 |
| C-20:1 | 1.53 | 2.94 |
| C-22 | 0.35 | 0.86 |
| C-22:1 | 0.30 | 0.33 |
| C-24 | 0.19 | 0.45 |
| Others | 0.57 | 11.23 |

### Example 3

### General Application of a Sulphated Oil

As a general treatment, approximately 2 quarts of the pH adjusted sulphated canola oil from Example 1 (“Sulplex”) was diluted with about 100 gallons of water and then sprayed onto plants covering a one-acre field.

### Example 4

### Sulplex Application on Hops (Humulus spp.)

This test was conducted on approximately 2 acres of Tomohawk™ variety (Humulus lupulus) which is extremely susceptible to powderly mildew (“PM”). A Rears sprayer was used and water was applied at the rate of 100 gallons per acre on all applications. The first application date was June 23 at the rate of 1 quart of Sulplex per acre. All subsequent applications were made at the rate of 1.75 quarts per acre. Application dates were 6/23, 7/6, 7/17, 7/26, 8/10 and 8/20. Leaf and cone infection counts were taken on August 18. Counts on the test plot were compared with a standard PM control program using the commercial fungicide Rally™ (Dow), implemented in the same field as the test plot. The raw data is shown in Tables 1-4 below.

TABLE 1
Number of infected cones out of 10 cones per plant detected in 4 test
plots (“TP”) using Sulplex (10 plants/plot)

| | | | | | | | | | | | Total | % |
|---|---|---|---|---|---|---|---|---|---|---|---|---|
| | | | | | | | | | | | infected | infected |
| TP1 | 3/10 | 4/10 | 2/10 | 1/10 | 1/10 | 3/10 | 1/10 | 2/10 | 2/10 | 1/10 | 20/100 | 20 |
| TP2 | 2/10 | 3/10 | 1/10 | 3/10 | 2/10 | 3/10 | 0/10 | 4/10 | 5/10 | 4/10 | 27/100 | 27 |
| TP3 | 4/10 | 5/10 | 1/10 | 1/10 | 4/10 | 1/10 | 1/10 | 0/10 | 2/10 | 0/10 | 19/100 | 19 |
| TP4 | 3/10 | 3/10 | 2/10 | 5/10 | 5/10 | 2/10 | 2/10 | 0/10 | 1/10 | 0/10 | 23/100 | 23 |
| | | | | | | | | | | | 89/400 | 22.25 |

TABLE 2
Number of infected cones out of 10 cones per plant detected in 4 test
plots (“TP”) using Rally ™ (10 plants/plot)

| | | | | | | | | | | | Total | % |
|---|---|---|---|---|---|---|---|---|---|---|---|---|
| | | | | | | | | | | | infected | infected |
| TP1 | 1/10 | 1/10 | 1/10 | 1/10 | 1/10 | 1/10 | 1/10 | 2/10 | 2/10 | 2/10 | 13/100 | 13 |
| TP2 | 5/10 | 8/10 | 4/10 | 4/10 | 2/10 | 2/10 | 4/10 | 3/10 | 4/10 | 3/10 | 39/100 | 39 |
| TP3 | 2/10 | 4/10 | 3/10 | 4/10 | 1/10 | 1/10 | 2/10 | 7/10 | 3/10 | 2/10 | 29/100 | 29 |
| TP4 | 4/10 | 3/10 | 5/10 | 5/10 | 4/10 | 2/10 | 3/10 | 2/10 | 9/10 | 3/10 | 40/100 | 40 |
| | | | | | | | | | | | 121/400 | 30.25 |

TABLE 3
Number of infected leaves per plant detected in 4 test plots (“TP”) using
Sulplex (10 plants/plot)

| | | | | | | | | | | | Total | % |
|---|---|---|---|---|---|---|---|---|---|---|---|---|
| | | | | | | | | | | | infected | infected |
| TP1 | 2/16 | 2/14 | 1/16 | 3/16 | 9/23 | 9/16 | 1/36 | 3/15 | 1/7 | 2/14 | 33/173 | 19.08 |
| TP2 | 3/28 | 5/22 | 6/25 | 5/13 | 2/11 | 10/30 | 4/13 | 2/13 | 3/15 | 3/16 | 43/186 | 23.12 |
| TP3 | 11/23 | 9/21 | 9/16 | 6/19 | 1/19 | 1/18 | 7/18 | 5/22 | 8/24 | 4/10 | 61/190 | 32.11 |
| TP4 | 2/28 | 13/24 | 5/11 | 4/16 | 1/10 | 4/14 | 1/19 | 4/13 | 5/15 | 5/18 | 44/168 | 26.19 |
| | | | | | | | | | | | 181/717 | 25.12 |

TABLE 4
Number of infected leaves per plant detected in 4 test plots (“TP”) using
Rally ™ (10 plants/plot)

| | | | | | | | | | | | Total | % |
|---|---|---|---|---|---|---|---|---|---|---|---|---|
| | | | | | | | | | | | infected | infected |
| TP1 | 5/19 | 5/23 | 5/11 | 1/11 | 2/24 | 11/21 | 7/26 | 3/11 | 2/19 | 11/21 | 52/186 | 27.96 |
| TP2 | 6/11 | 14/30 | 3/11 | 10/29 | 3/31 | 3/20 | 9/20 | 4/13 | 5/16 | 5/17 | 62/198 | 31.31 |
| TP3 | 3/12 | 9/20 | 10/24 | 12/20 | 3/15 | 11/42 | 5/24 | 9/16 | 12/20 | 8/18 | 82/211 | 38.86 |
| TP4 | 8/27 | 5/25 | 4/21 | 2/14 | 5/14 | 5/10 | 11/18 | 10/20 | 9/17 | 7/10 | 66/176 | 37.50 |
| | | | | | | | | | | | 262/771 | 33.91 |

Comparison of the results from Tables 1 and 2 indicate that only 22.25% of the hop cones exhibited signs of infection by powdery mildew after using Sulplex, compared to 30.25% of the cones after using Rally™, representing a 26% increase in effective control over a commercial fungicide. Comparison of the results from Tables 3 and 4 indicate that only 25.12% of the hop leaves exhibited signs of infection by powdery mildew after using Sulplex, compared to 33.91% of the leaves after using Rally™, again representing a 26% increase in effective control.

### Example 5

### Association of Analytical Communities (AOAC) Germicidal and Detergent Sanitizer Test Using Sulplex

Raspberry mold was cultured for 48 hours at 35° C. to establish proper growth. The Sulplex liquid formula was first diluted 64×(1.56%) and then introduced into the infected medium. After 30 seconds, the medium was then neutralized with phosphate buffer water and analyzed. The Sulplex solution was observed to kill 99.999% of the mold within the 30-second time frame.

### Example 6

### Sulphur-Containing Oils for Promoting Uptake of Nutrients by a Plant

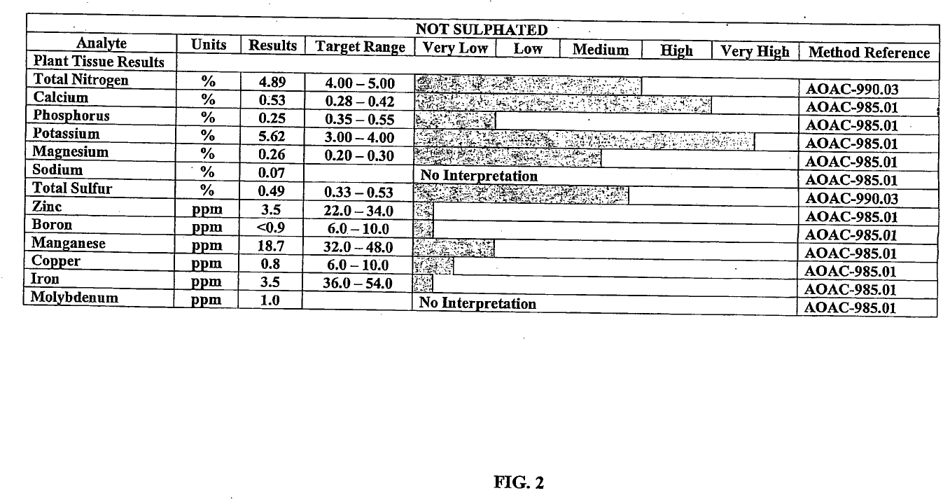

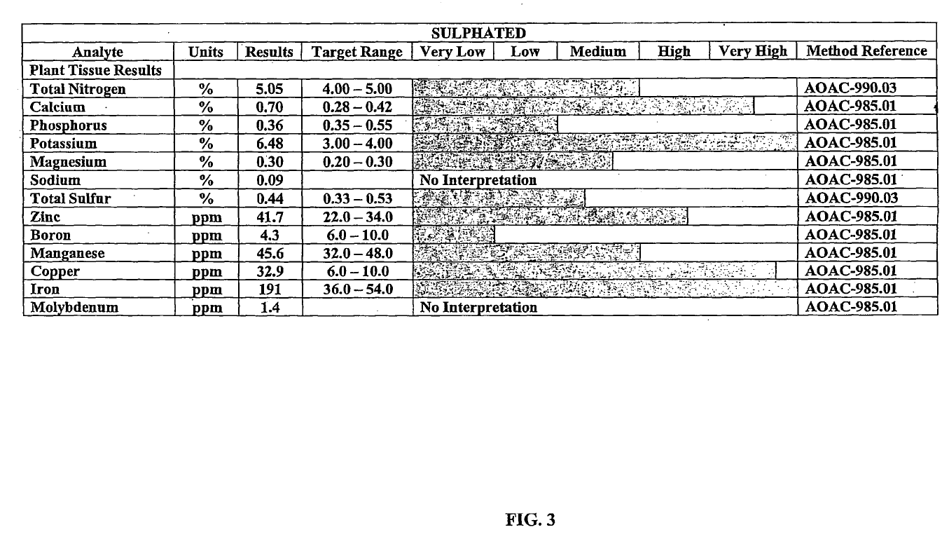

An analysis of the tissue of barley at the 4-6 leaf stage untreated with Sulplex is shown in FIG. 2. An analysis of the tissue of barley at the 4-6 leaf stage treated with Sulplex is shown in FIG. 3 for comparison. While there were relatively small increases in the nitrogen, calcium, phosphorus, potassium, magnesium and sodium content in the tissue of the treated plants, there was a dramatic increase in the amount of zinc, boron, manganese, copper and iron when compared to the untreated plants. Visually, the treated plants appeared robust and, unlike the untreated plants, were not losing significant amounts of water through transpiration.

### Example 7

### Rat Toxicity Trial Using Sulplex

The water consumption of a mature male hooded rat (275 g) receiving an ad libitum ration of hog grower pellets was measured over a period of 5 days and was found to be 18.6 g per day. The water was then replaced by a 0.5% v/v dispersion of the original Sulplex. Water consumption was again measured and was found to be 27.3 g per day over a 96 hour period. Apart from the increased fluid consumption during the test period, the rat's behavior and condition appeared normal, suggesting that there was no indication of toxicity due to the Sulplex being added to the water.

### Example 8

### Chinook Toxicity Trial Using Sulplex

A feeding trial was set up using two 1600 liter tanks containing approximately 400 20 g Chinook each. The control group received a commercial pelleted grower ration (Taplow Feeds Ltd) while the test tank received the same ration to which the original Sulplex was admixed at the rate of 10% w/w. The water temperature was 10° C. and the water in the tanks exchanged at the rate of one change per hour. Dissolved oxygen was maintained at greater than 7.5 ppm in the tank outlet overflows. The fish were fed three times daily. Although the intention was to feed to satiation at each feeding, the results suggested a tendency of the fish feeders to feed the same amount of feed to each tank at each feeding. The trial began on May 2 and was terminated on August 4, after 94 days, at which time the fish were killed, counted and weighed as a group. There was no mortality over the trial period in the Sulplex treatment tank and only 2 moralities (due to gill fungus) in the control tank. At the end of the trial, the test tank contained 424 fish having a total weight of 19.52 kg (mean weight of 46.04 g), while the control group contained 355 fish having a total weight of 19.38 kg (mean weight 54.59 g). The difference in final numbers was essentially due to a difference in the starting numbers. Post mortem internal examination of fish from each group at the end of the trial revealed no difference in the excellent condition of the two groups. There was no indication of toxicity of Sulplex to the fish when ingested in feed. The 10% level of inclusion in feed was the amount of Sulplex which would soak into the feed and still allow the mixture to be handled as dry feed. It was evident that in the processes of feeding, ingestion and defecating, the amount of Sulplex which dispersed into the tank water was not sufficient to cause any toxic effects on the fish gills. The difference in final individual weights between the two groups was probably due to an artifact of the feeding technique which resulted in a marginal underfeeding per fish due to the slightly greater number of fish in the Sulplex tank.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications cited herein are incorporated herein by reference for the purpose of disclosing and describing specific aspects of the invention for which the publication is cited.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will appreciate that various modifications can be made in the present invention without departing from the spirit or scope of the invention. Thus, it is intended that the present invention cover the modifications and variations of this invention provided they come within the scope of the appended claims and their equivalents.

We claim:

1. A process of preparing a water-soluble sulphur-containing oil, comprising the steps of:
combining an oil containing at least one sulphur-reactive site selected from the group consisting of an alkene bond, an alkyne bond and a hydroxy group with a sulphur-donating agent under conditions sufficient to cause at least about 30% of the sulphur-reactive sites in the oil to react with the sulphur-donating agent,
allowing the resulting sulphur-containing oil to stand for a time sufficient to complete reaction with the sulphur-donating agent; and
neutralizing the sulphur-containing oil with a base.

2. The process according to claim 1, wherein the sulphur-donating agent is selected from the group consisting of sulphuric acid, sulphur trioxide and sodium bisulfite.

3. The process according to claim 2, wherein the sulphur-donating agent is sulphuric acid.

4. The process according to claim 3, wherein the concentration of the sulphuric acid is at least about 2 N.

5. The process according to claim 1, wherein the sulphur-containing oil is selected from the group consisting of a sulphated oil, a sulphonated oil and mixtures thereof.

6. The method according to claim 5, wherein the oil is a sulphated oil.

7. The process according to claim 1, wherein the base is selected from the group consisting of sodium hydroxide, potassium hydroxide, sodium carbonate, potassium carbonate, calcium hydroxide, diethanolamine and ammonium hydroxide.

8. The process according to claim 7, wherein the base is sodium hydroxide.

9. The process according to claim 8, wherein the concentration of the sodium hydroxide is at least about 2 N.

10. The process according to claim 1, wherein the oil is selected from the group consisting from canola, coconut, palm, cottonseed, palm kernel, olive, flax, castor, soybean, sunflower, corn, grape seed and peanut.

11. The process according to claim 10, wherein the oil is canola oil.

12. The process according to claim 1, wherein the oil and the sulphur-donating agent are combined under cooling.

13. The process according to claim 12, wherein the cooling maintains the temperature below about 40° C.

14. The process according to claim 1, wherein the neutralization of the sulphur-containing oil with the base occurs under cooling.

15. The process according to claim 14, wherein the cooling maintains the temperature below about 40° C.

16. A fungicidal, water-soluble sulphur-containing oil prepared by
combining an oil containing at least one sulphur-reactive site selected from the group consisting of an alkene bond, an alkyne bond and a hydroxy group with a sulphur-donating agent under conditions sufficient to cause at least about 30% of the sulphur-reactive sites in the oil to react with the sulphur-donating agent,
allowing the resulting sulphur-containing oil to stand for a time sufficient to complete reaction with the sulphur-donating agent; and
neutralizing the sulphur-containing oil with a base.

17. The sulphur-containing oil according to claim 16, wherein the sulphur-donating agent is selected from the group consisting of sulphuric acid, sulphur trioxide and sodium bisulfite.

18. The sulphur-containing oil according to claim 17, wherein the sulphur-donating agent is sulphuric acid.

19. The sulphur-containing oil according to claim 16, wherein the base is sodium. hydroxide.

20. The sulphur-containing oil according to claim 19, wherein the concentration of the sodium hydroxide is at least about 2 N.

21. The sulphur-containing oil according to claim 18, wherein the oil is canola oil.

22. A method of controlling or killing a plant pathogen present in a plant, in soil, in water or on an artificial surface comprising contacting the pathogen with an effective amount of the sulphur-containing oil of claim 16.

23. The method according to claim 22, wherein the pathogen is selected from the group consisting of molds, mildews, fungi, mosses and rusts.

24. The method according to claim 22 wherein the artificial surface is a roof.

25. A method of stimulating a plant's uptake of nutrients from the soil, comprising treating a plant in need thereof with an effective amount of the sulphur-containing oil of claim 16.

26. A method of adjusting soil pH comprising treating the soil with an amount of the sulphur-containing oil of claim 16 effective to obtain a contemplated pH.
